(19) United States
(12) Patent Application Publication
(10) Pub. No.: US 2005/0124690 A1
(43) Pub. Date: Jun. 9, 2005

(54) Pharmaceutical composition for the treatment of seborrhea containing 4-hydroxy-5-methoxy-4-[2-methyl-3(3-methyl-2-butenyl)-2-oxiranyl]-1-oxaspiro[2,5]octan-6-one

(76) Inventors: Cheol-Sik Yoon, Gyeonggi-do (KR); Hyun-Soo Park, Gyeonggi-do (KR); Chan-Won Park, Seoul (KR)

Correspondence Address: MICHAEL BEST & FRIEDRICH, LLP, ONE SOUTH PINCKNEY STREET, P O BOX 1806, MADISON, WI, 53701, US

(21) Appl. No.: 10/514,373

(22) PCT Filed: May 17, 2002
(86) PCT No.: PCT/KR02/00931

(30) Foreign Application Priority Data
May 13, 2002 (KR) 2002-0026280

(51) Int. Cl. A61K 31/335; A61K 7/00

(52) U.S. Cl. 514/462; 424/401

(57) ABSTRACT

Disclosed is a pharmaceutical composition containing a pharmaceutically effective amount of 4-hydroxy-5-methoxy-4-[2-methyl-3-(methyl-2-butenyl)-2-oxiranyl]-1-oxaspiro[2,5]octan-6-one, which is capable of preventing or treating seborrhea caused by a fungus, Pityrosporum ovale.

### TECHNICAL FIELD

The present invention relates to a pharmaceutical composition for the prevention or treatment of seborrhea caused by a fungus, Pityrosporum ovale, which contains a pharmaceutically effective amount of 4-hydroxy-5-methoxy-4-[2-methyl-3-(3-methyl-2-butenyl)-2-oxiranyl]-1-oxaspiro[2,5]octan-6-one.

### BACKGROUND ART

In general, dandruff itself, which frequently occurs on the scalp, is not associated with inflammation, but is just a physiological material. That is, dandruff, which is scale-shaped, is dried keratin stripped off from the scalp. However, as a kind of dermatitis, dandruff is a condition where seborrheic dermatitis occurs on the scalp with no severe symptoms, and is not contagious. Typically, dandruff starts to appear in a small area on the scalp at the initial stage, and gradually spreads to the entire scalp, and in the worst cases, it becomes thick, accompanied by crusting and erythema and even weeping fluid upon intensive scratching.

The exact etiology of dandruff has not been established, but there are various factors, which are presumed including genetic factors, stress, hormones (especially, androgen), habits, fungi, administration of drugs and foods. A lipophilic pleomorphic fungus, Pityrosporum ovale has been believed to be most influential. It inhabits in the scalp and excessively proliferates to increase its population about 10 to 20 times, causing dandruff (Bernardidson, Dandruff: Cause and Control, Drug and Cosmetic industry, 96, 636-, May 1965; James J. Leyden, et. al., Role of Microorganisms in Dandruff, Arch Dermatol, 112, 333-338, March 1976; Norman J. Van Abbe, et: al., Dandruff: Infection, International Journal of Cosmetic Science, 8, 37-44, 1986; and Sam Shuster, Dandruff, Seborrhoeic Dermatitis, and Pityrosporum ovale, Cosmetics and Toiletries, 103, 87-91, March 1988).

As described above, dandruff is a common form of seborrhoeic dermatitis, which is limited to the scalp and accompanies with no severe symptoms. Also, dandruff can extend to other regions of the body, and this is called seborrhoeic dermatitis. Like the case of dandruff, no certain cause of seborrhoeic dermatitis is known, but, considering its occurrence in skin areas with developed sebaceous glands, it is considered to be associated with the over-secretion of sebum in sebaceous glands as well as to be affected by hormones, owing to its rare appearance before adolescence. Also, the excessive growth of Pityrosporum ovale is believed to be another etiology of seborrhoeic dermatitis, for the reason that Pityrosporum ovale has been detected on the scalp of many patients suffering from seborrhoeic dermatitis, and antifungal agents relieve dermatitis lesions by reducing the viability of the fungus.

It is well known that the fungus, P. ovale, inhabits all human beings, especially skin areas having many sebaceous glands, such as the scalp, causing itchy dandruff. In addition, upon over-proliferating, the fungus, P. ovale may spread to eyebrows, eyelids, nasolabial folds, lips, ears, regions of sternum, armpits, regions under breasts, navels, groins, or wrinkled regions between buttocks, and cause seborrhoeic dermatitis thereat. Further, the fungus P. ovale is mainly observed after adolescence during which time sebaceous glands develop rapidly, but not before the adolescence.

There are many externally applied agents to treat skin conditions caused by the fungus, P. ovale, as disclosed in the following patents. European Pat. No. 819427 discloses a cosmetic and/or skin pharmaceutical composition for treating seborrhoeic dermatitis using 2-(3-iodo-2-propynyl)-buthylcarbamate. Also, a detergent composition containing 2-mercaptoquinoxaline-1-oxides, salts thereof, and 2-(1-oxoquinoxalinyl) disulfides for the treatment of dandruff is disclosed in U.S. Pat. Nos. 3,971,725 and 3,852,443. U.S. Pat. No. 4,472,421 is claimed shampoo composition for treating skin conditions caused by Pityrosporum ova/e using azole compounds. Also, described in Japanese Pat. Laid-open Publication No. Heisei. 4-164,013 is a composition for inhibiting the growth of Pityrosporum ovale, and treating cutaneous and scalp diseases caused by the fungus, which contains capable as an active ingredient.

In addition, well known are shampoo compositions containing zinc pyridinethione, which is capable of effectively inhibiting the proliferation and activity of the fungus, P. ovale, and thus normalizing the metabolic activity of cells in the scalp (refer to U.S. Pat. No. 2,809,971; U.S. Pat. No. 3,236,733; U.S. Pat. No. 3,753,196; Japanese Pat. Laid-open Publication No. Sho. 52-60810; U.S. Pat. No. 4,323,683; U.S. Pat. No. 4,345,080; U.S. Pat. No. 4,379,753; U.S. Pat. No. 4,470,982; and European Pat. No. 285,388).

Moreover, various drugs, non-drug products and cosmetics are known as agents capable of preventing and/or treating pathological changes of the skin caused by Pityrosporum ovale, and these are also on sale. However, the effect of these products is not satisfactory, and in most cases, they have a very low effect or a high toxicity. For example, itraconazol and ketoconazol, which are synthetic organic compounds, are mainly used as therapeutic materials for seborrhoeic dermatitis caused by Pityrosporum ova/e, but their frequency of use is limited owing to their high toxicity and severe side effects.

On the whole, despite the existence of many prior arts and products on sale for the prevention, improvement, and treatment of skin conditions caused by the fungus, P. ovale, there is a need for a new agent having high antifungal activity against P. ovale, as well as no toxicity.

### DISCLOSURE OF INVENTION

Leading to the present invention, the intensive and thorough research for an agent having antifungal activity against Pityrosporum ovale conducted by the present inventors with an aim to solve the problems encountered in prior arts resulted in the finding that a compound, 4-hydroxy-5-methoxy-4-[2-methyl-3-(3-methyl-2-butenyl)-2-oxiranyl]-1-oxaspiro[2,5]octan-6-one, has a high antifungal activity against Pityrosporum ovale while having no toxicity, and a composition containing the compound, 4-hydroxy-5-methoxy-4-[2-methyl-3-(3-methyl-2-butenyl)-2-oxiranyl]-1-oxaspiro[2,5]octan-6-one, as an active ingredient has a practical and remarkable effect on skin conditions overall or those partially caused by Pityrosporum ovale.

In an aspect of the present invention, there is provided a pharmaceutical composition useful for the prevention and treatment of skin conditions caused by a fungus, Pityrosporum ovale, which is characterized by containing a pharmaceutically effective amount of a compound, 4-hydroxy-5-methoxy-4-[2-methyl-3-(3-methyl-2-butenyl)-2-oxiranyl]-1-oxaspiro[2,5]octan-6-one.

In another aspect of the present invention, there is provided a method of preventing or treating skin conditions caused by a fungus, Pityrosporum ovale, which is characterized by applying a pharmaceutical composition containing a pharmaceutically effective amount of a compound, 4-hydroxy-5-methoxy-4-[2-methyl-3-(3-methyl-2-butenyl)-2-oxiranyl]-1-oxaspiro[2,5]octan-6-one, to skin of patients suffering from seborrhea.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a photograph showing conidia of Metarhizium anisopliae var. anisopliae CS1448 strain; and

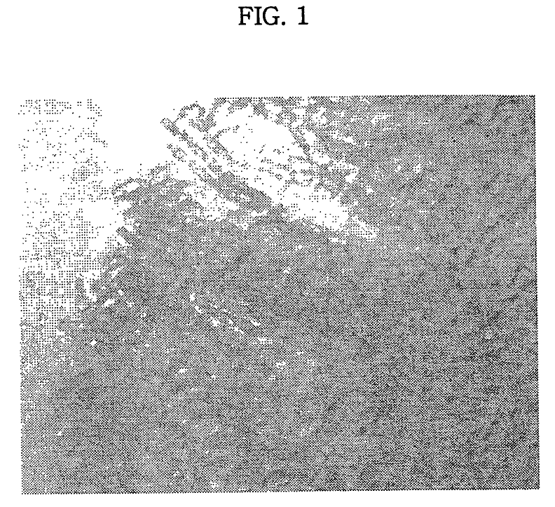

FIG. 2 is a photograph showing an antifungal effect and minimum inhibitory concentration of 4-hydroxy-5-methoxy-4-[2-methyl-3-(3-methyl-2-butenyl)-2-oxiranyl]-1-oxaspiro[2,5]octan-6-one against Pityrosporum ovale.

### BEST MODE FOR CARRYING OUT THE INVENTION

Definition of Terms

The term “pharmaceutically effective amount”, as used herein, means the amount of a pharmaceutical composition, which is effective for the prevention or treatment of one or more skin conditions, upon its application to skin.

The term “pharmaceutical composition”, as used herein, refers to pharmaceutical preparations, detergents, or cosmetics containing a pharmaceutically effective amount of 4-hydroxy-5-methoxy-4-[2-methyl-3-(3-methyl-2-butenyl)-2-oxiranyl]-1-oxaspiro[2,5]octan-6-one as an active ingredient for the prevention and treatment of skin conditions caused by a fungus, Pityrosporum ovale.

The term “skin conditions”, as used herein, refers to conditions present on any region of skin affected by Pityrosporum ovale, and includes conditions considered as cutaneous diseases as well as those not considered as cutaneous diseases. The fungus, P. ovale, inhabits the uppermost skin layers of humans, and spreads by budding. The fungus may cause changes in skin including pityriasis simplex, pityriasis oleosa, and pityriasis circinata, which are not considered as cutaneous diseases. In addition, the fungus can cause cutaneous diseases, such as seborrhoeic dermatitis or acne culgaris.

Typically, all of skin conditions caused or partially caused by the fungus, P. ovale are called “seborrhea”. Therefore, the term “seborrhea”, as used herein, includes all kinds of cutaneous diseases, such as seborrhoeic dermatitis or acne vulgaris, and non-cutaneous diseases, such as pityriasis simplex, pityriasis oleosa or pityriasis circinata. Since dandruff is typically defined a condition where the occurrence of seborrhoeic dermatitis is confined to the scalp, the term “seborrhea” also includes dandruff.

The term “treatment”, as used herein, means a result of application of the pharmaceutical composition of the present invention to skin having seborrhea, where the complete recovery of symptoms of seborrhea includes partial recovery, improvement, and alleviation.

The term “prevention”, as used herein, means that symptoms of seborrhea do not develop, due to inhibition or prevention of infection and growth of Pityrosporum ovale through application of the pharmaceutical composition of the present invention to the skin, especially the scalp.

### Active Compound, 4-hydroxy-5-methoxy-4-[2-methyl-3-(3-methyl-2-butenyl)-2-oxiranyl]-1-oxaspiro[2,5]octan-6-one

A compound, 4-hydroxy-5-methoxy-4-[2-methyl-3-(3-methyl-2-butenyl)-2-oxiranyl]-1-oxaspiro[2,5]octan-6-one, which is used as an active ingredient in the pharmaceutical composition of the present invention, is represented by the chemical formula 1, below.

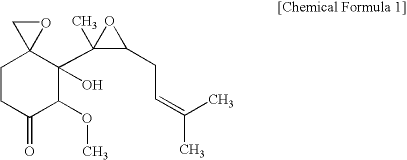

The compound, 4-hydroxy-5-methoxy-4-[2-methyl-3-(3-methyl-2-butenyl)-2-oxiranyl]-1-oxaspiro[2,5]octan-6-one, represented by the chemical formula 1, is a derivative of oxaspiro[2,5]octane, and can be prepared by chemical synthesis methods as disclosed in literatures (E. J. Corey et al., J. Am. Chem. Soc., 1985, 107:256-257; E. J. Corey et al., J. Am. Chem. Soc., 1994, 116:12109-12110).

As a derivative of oxaspiro[2,5]octane, 4-hydroxy-5-methoxy-4-[2-methyl-3-(3-methyl-2-butenyl)-2-oxiranyl]-1-oxaspiro[2,5]octan-6-one can be used as a pharmaceutical material, as described in literatures (E. J. Corey et al., J. Am. Chem. Soc., 1985, 107:256-257; E. J. Corey et al., J. Am. Chem. Soc., 1994, 116:12109-12110), exhibiting antitumor and immuosuppressive activity.

However, until now, there has been no report that the compound, 4-hydroxy-5-methoxy-4-[2-methyl-3-(3-methyl-2-butenyl)-2-oxiranyl]-1-oxaspiro[2,5]octan-6-one, is used for the treatment of seborrhea, dandruff, and other diseases caused by the fungus, P. ovale.

### Pharmaceutical Preparation Containing 4-hydroxy-5-methoxy-4-[2-methyl-3-(3-methyl-2-butenyl)-2-oxiranyl]-1-oxaspiro[2,5]octan-6-one

In accordance with an aspect of the present invention, there is provided a pharmaceutical preparation containing 4-hydroxy-5-methoxy-4-[2-methyl-3-(3-methyl-2-butenyl)-2-oxiranyl]-1-oxaspiro[2,5]octan-6-one as an active ingredient, which is capable of preventing or treating skin conditions caused by Pityrosporum ovale. The pharmaceutical preparation can be formulated as ointments, creams, pastes, lotions, liniments, external liquid solutions, tinctures, glycerogelatins, external powders, aerosols, plasters, and the like. Such formulations are described in a book, which is well known in the pharmaceutical field (Remington's Pharmaceutical Science, 15th Edition, 1975. Mack Publishing Company, Easton, Pa. 18042, Chapter 87: Blaug, Seymour).

According to the present invention, an ointment is provided as a preferable formulation. The ointment of the present invention can be prepared from carbohydrate bases, which may be exemplified by petrolatum, white petrolatum, yellow ointment and mineral oil; absorbent bases, which may be exemplified by hydrophilic petrolatum, anhydrous lanolin, lanolin and cold cream; water washable bases such as hydrophilic ointment; or water-soluble bases such as polyethylene glycol ointment. The selection and preparation of the bases may be achieved depending on various factors including the release rate of a drug from a base, the effect of a base on enhancement of percutaneous adsorption, the moisture sealing effect of a base on water in skin, the stability of a drug in a base and the influence of a drug on a base, and is a common skill of experts in the pharmaceutical preparation field.

According to the present invention, a cream is provided as a preferable formulation. Examples of the cream according to the present invention include water in oil (w/o) types, such as cold creams and emollient creams; and oil in water (o/w) types, which may be exemplified by shaving creams, vanishing creams, hand creams and cleansing creams. More preferable are vanishing creams, which typically contain water and stearic acid. Patients and doctors prefer a cream form to an ointment form because o/w creams are easier into wash off than ointments. In view of this fact, in the present invention, a cream form is preferable.

According to the present invention, a lotion is provided as a preferable form. A lotion may be prepared in the form of suspension, emulsion or solution, and this preparation is a common skill of experts in the pharmaceutical preparation field. In the present invention, more preferable is a white lotion, which may be prepared by dissolving sulfated potash in water to a content of 4% and then filtering, and adding a solution of zinc sulfate to the solution with gentle agitation at a constant velocity.

According to the present invention, a liniment is provided as a preferable formulation. A liniment may be prepared by any of oil liniments and ethanol liniment. More preferable is an oil liniment causing little irritation to skin. Examples of the oil liniment include non-volatile oils, which may be exemplified by almond oils, peanut oils, cottonseed oils, etc., mixtures of non-volatile oils and volatile oils, which may be exemplified by wintergreen, turpentine, etc.

### Preparation of Detergent and Cosmetics Containing 4-hydroxy-5-methoxy-4-[2-methyl-3-(3-methyl-2-butenyl)-2-oxiranyl]-1-oxaspiro[2,5]octan-6-one

In accordance with an aspect of the present invention, there are provided detergents and cosmetics containing 4-hydroxy-5-methoxy-4-[2-methyl-3-(3-methyl-2-butenyl)-2-oxiranyl]-1-oxaspiro[2,5]octan-6-one as an active ingredient, which is capable of preventing or treating skin conditions caused by Pityrosporum ovale. The pharmaceutical composition according to the present invention can be formulated as various detergents and cosmetics, but are not limited thereto, including hair toiletry, hairdressing, and skin toiletry. More particularly, the formulations of the detergent and cosmetics may exemplified by hair soaps, hair creams, aqueous and aqueous alcoholic hair lotions, wave-setting lotions (hair fixer), hairdressing creams and gels, hair sprays, hair tonics, hair oils, hair pomades, hair brilliants, and especially, hair rinses and shampoos. Examples of the skin toiletry include soap and cleansing compositions in the form of solid, liquid, or power, liquid creams and skin gels, skin oils, face lotions, astringents and deodorants.

Preferably, according to the present invention, the detergent and cosmetics compositions containing 4-hydroxy-5-methoxy-4-[2-methyl-3-(3-methyl-2-butenyl)-2-oxiranyl]-1-oxaspiro[2,5]octan-6-one as an active ingredient may include an auxiliary agent, which is selected from the group consisting of surfactants, stabilizers, preservatives, moisturizers, anti-inflammatory agents, anti-oxidants, coloring agents, water and mixtures thereof. The auxiliary agent can be contained in an amount of about 98.0 to 99.1 wt % of the composition.

The surfactant useful in preparation of the detergents and cosmetics of the present invention may be present in an anionic, a cationic or a zwitterionic form, typically, contained in an amount of at least 30 wt %, and preferably, at least 70 wt %. Those skilled experts in the art can easily determine the kind and amount of the surfactant.

The stabilizer useful in preparation of the detergents and cosmetics of the present invention is, preferably, glycol stearate, but is not limited to this. The stabilizer is typically contained in an amount of about 0.1 to 5 wt % of the composition.

Preferably, the preservative useful in preparation of the detergents and cosmetics of the present invention includes, but is not limited to, tetrasodium ethylenediamine tetraacetate (tetrasodium EDTA), 1-(3-chloroaryl)-3,5,7-traaza-1-adamanthane, parabene, methyl parabene, a mixture of 5-chloro-2-methyl-4-isothiazoline-3-one and 2-methyl-4-isothiazoline-3-one, phenoxyethanol, benzylalcohol, benzophenone-4, methylchloroisothiazolinone, methylisothiazolinone and mixtures thereof.

The preservative is, when used, typically contained in about 0.01 to 6 wt % of the composition, preferably, about 0.05 to 4 wt %, and more preferably, about 0.1 to 2 wt %.

Preferably, the moisturizer useful in preparation of the detergents and cosmetics of the present invention includes wheat proteins (e.g., laurdimonium hydroxypropyl, hydrolyzed wheat protein), hair keratin amino acids, sodium peroxylin carbolic acid, pantenole, tocopherol (vitamin E), dimethicone and mixtures of thereof. Also, the moisturizers, especially hair keratin amino acids, can contain sodium chloride. The moisturizer is typically used in about 0.01 to 10 wt % of the composition, preferably, abut 0.05 to 1.5 wt %, and more preferably, abut 0.1 to 1 wt %.

Preferably, the coloring agent useful in preparation of the detergents and cosmetics of the present invention includes FD&C green No. 3, Ext. D&C violet No. 2, FD&C yellow No. 5, FD&C red No. 40 and mixtures thereof, and is, when used, typically used in an amount of about 0.001 to 0.1 wt % of the cleansing and cosmetic composition, and more preferably, abut 0.005 to 0.05 wt %.

As the anti-inflammatory agent useful in preparation of the detergents and cosmetics of the present invention, preferable is an anti-inflammatory agent suitable for local administration and being pharmaceutically permeable, and most preferable is alantonin. The anti-inflammatory agent may be, when used, used in an amount sufficient to inhibit or alleviate inflammation, typically about 0.1 to 2 wt % of the composition, preferably, about 0.3 to 1.5 wt %, and more preferably, about 0.4 to 1 wt %.

Anti-oxidants of both enzymatic and non-enzymatic types may be used in the detergent and cosmetic preparations of the present invention. Examples of natural enzymatic anti-oxidants include superoxide dismutase (SOD), catalase, and glutathione peroxidase, and suitable non-enzymatic anti-oxidants include vitamin E (e.g., tocopherol), vitamin C (ascorbic acid), carotenoides, echinacoside and cafeoyl derivatives, oligomeric proanthocyanidins or proanthanols (e.g., grape seed extract), silymarin (e.g., milk thistle extract, Silybum marianum), gingko biloba, green tea polyphenyls, and the like and mixtures thereof. Carotenoids are powerful anti-oxidants, and they include beta-carotene, canthaxanthin, zeaxanthin, lycopen, lutein, crocetin, capsanthin and the like. Preferably, the anti-oxidant component includes Vitamin E, Vitamin C or a carotenoid. The anti-oxidant component, when used, is present in an amount sufficient to inhibit or reduce the effects of free radicals at the scalp. The anti-oxidant component may be present in an amount from about 0.001 to 1 wt %, preferably from about 0.01 to 0.5 wt % of the composition.

Also, the detergent and cosmetic compositions may contain other auxiliary agents well known to those skilled experts in the art. Examples of the auxiliary agents include perfumes, pigments, which include all those coloring hair concurrently or separately supplying color, solvents, opacificers or pearlescent agents (e.g., ester of fatty acid and polyol, magnesium salt and iron salt of fatty acid), copolymer dispersions, thickeners (e.g., sodium chloride, potassium chloride, ammonium chloride, sodium sulfate), alkylolamide fatty acids, cellulose derivatives, natural gums, plant extracts, albumin derivatives (e.g., gelatin), collagen hydrolysis products, natural or synthesized polypeptides, yolks, lecithin, lanoline and its derivatives, fats, oils, fatty alcohols, silicon, deordarant, antimicrobial agents, anti-seborrhea agents, keratolytic agents (e.g., sulfur, salicylic acid, benzoyl peroxide, selenium sulfide, PG, FA, enzymes) and keratinizing agents (e.g., tar, sulfur, salicylic acid, selenium sulfide, antimicrobial agents, benzoyl peroxide, chlorohexidine, iodine, trichloric acid, antifungal agents, enzymes).

Pharmaceutically Effective Amount

The magnitude of a prophylactic or therapeutic dose of the pharmaceutical preparation, cleansing and cosmetic preparations according to the present invention, may vary depending on the severity of the condition to be treated and their formulations. Also, the dose, namely, the dose frequency, may vary according to the age, body weight, and response of patients. Generally, the compound used as an active ingredient in the pharmaceutical composition of the present invention, 4-hydroxy-5-methoxy-4-[2-methyl-3-(3-methyl-2-butenyl)-2-oxiranyl]-1-oxaspiro[2,5]octan-6-one, can be present in the preparation described above in an amount of 0.01 to 10 wt %, preferably, 0.1 to 2 wt %, and more preferably, 0.9 to 1 wt %. Within the range, the content of the compound in a specific preparation may be determined according to the use of the preparation. In addition, a specific preparation, such as a concentrated preparation, which should be diluted before use, may contain a much higher content of the compound.

All of the preparations according to the present invention may be manufactured using ordinary skill in the art by mixing 4-hydroxy-5-methoxy-4-[2-methyl-3-(3-methyl-2-butenyl)-2-oxiranyl]-1-oxaspiro[2,5]octan-6-one and each component, and formulating the mixture in a suitable form. In accordance with the present invention, various preparations containing 4-hydroxy-5-methoxy-4-[2-methyl-3-(3-methyl-2-butenyl)-2-oxiranyl]-1-oxaspiro[2,5]octan-6-one may be used according to typical methods, and preferably, by applying or massaging to the scalp and other skin areas afflicted with seborrheic dermatitis.

The Most Preferable Formulation

A shampoo is provided as the most preferable formulation of the pharmaceutical composition according to the present invention. A shampoo may be formulated in the form of transparent liquids, opaque liquids, gels, creams, or powders. The interactions of shampoo with hair, skin or scalp are dependent on the kind of surfactant, as a base of the shampoo, which may be an anionic, a cationic, a nonionic surfactant or a mixture thereof. Those of ordinary skill in the art can easily achieve the selection of the surfactant. The shampoo composition of the present invention, typically, comprises at least 30 wt % of surfactant, and preferably, at least 70 wt %.

The anionic surface active materials useful in the present invention may be exemplified by alkyl carboxylate and alkylene carboxylate, alkyl ether carboxylate, fatty alcohol sulfate, fatty alcohol-ether sulfate, alkylolamide sulfate and alkylolamide sulfonate, alkansulfonate and hydroxyalkane sulfonate, olefine sulfonate, acyl ester of isethionate, α-sulfo-fatty acid ester, alkylbenzensulfonate, alkylphenyl glycol ether-sulfonate, sulfosuccinate, sulfosuccinate half-ester and diester, fatty alcohol-ether phosphate, albumin-fatty acid condensates, alkyl monoglyceride sulfate and sulfonate, alkyl glyceride-ether sulfonate, fatty acid methyltauride, fatty acid sarcosinate and sulforysinolate. Herein, the alkyl and acyl groups contain from 10 to 20 carbon atoms. The compounds and mixtures of thereof can be used in the form of water-soluble or water-dispersible salts, which may exemplified by sodium, potassium, magnesium, ammonium, monoethanol ammonium, diethanol ammonium, triethanol ammonium, and similar alkylol ammonium salts.

Suitable cationic surfactants useful in the present invention include quaternary ammonium salts, which may exemplified by di-(C₁₀ to C₂₀-alkyl)dimethyl ammonium chloride or bromide, more preferably, di-(C₁₂ to C₁₈-alkyl)dimethyl ammonium chloride or bromide; C₁₀ to C₂₄-alkyl-dimethylethyl ammonium chloride or bromide; C₁₀ to C₂₄-alkyl-trimethylethyl ammonium chloride or bromide, more preferably, cethyl-trimethylammonium chloride or bromide and C₂₀ to C₂₂-alkyl-trimethyl ammonium chloride or bormide; C₁₂-C₁₈-alkyl-dimethylbenzyl ammonium chloride or bromide, more preferably, C₁₂ to C₁₈ alkyl-dimethylbenzyl ammonium chloride; N—(C₁₀ to C₁₈-alkyl)-pyridinium chloride or bromide, more preferably, N—(C₁₂ to C₁₆-alkyl)-pyridinium chloride or bromide; N—(C₁₂-C₁₈-alkyl)-isoquinolinium chloride, bromide, or monoalkyl-sulfate; N—(Cl₂-C₁₈-alkylolcholaminoformylmethyl)-pyridinium chloride; N—(C₁₂ to C₁₈-alkyl)-N-methyl-morpholinium chloride, bromide or monoalkylsulfate; N—(C₁₂ to C₁₈-alkyl)-N-ethyl-morpholinium chloride, bromide or monoalkylsulfate; C₁₆ to C₁₈ alkyl-pentaocetyl ammonium chloride; diisobutyl-penoxyethoxyethyl dimethylbenzyl ammonium chloride; hydrochloric acid of N,N-diethylaminoethyl-stearylamide and oleilamide, acetic, lactic, citric and phosphatic salts; N-acylamidoethyl-N,N-diethyl-N-methylammonium chloride, bromide or monoalkyl-sulfate, and N-acylamidoethyl-N,N-diethyl-N-benzylammonium chloride, bromide or monoalkyl-sulfate, wherein the acyl group is preferably stearyl or oleyl.

The non-ionic surfactants may be used with auxiliary agents in the shampoo composition of the present invention owing to its low foaming ability. The non-ionic surfactants include, but are not limited to, lyophilic high molecular weight esters of aliphatic multivalent alcohols and aliphatic polycarboxylic acids, and polyglycol ester of fatty acid, which may be exemplified by fatty alcohol ethoxylate (alkylpolyethylene glycol); alkylphenylpolyethylene glycol; alkylmercaptothane-polyethylene glycol; fatty amine ethoxylate (alkylamine-polyethylene glycol); fatty acid ethoxylate (acid-polyethylene glycol); fatty acid ethoxylate (acid-polyethylene glycol); polypropylene glycol ethoxylate (fluronic); fatty acid alkylolamide (fatty acid amidepolyethylene glycol); sucrose ester; sorbitol ester; and polyglycol ester.

The amphoteric surfactants useful in the shampoo composition of the present invention include alkali metal salts and mono-, di-, and tri-alkylolammonium salts, which may be exemplified by N—(C-₁₂-C₁₈-alkyl)-α-aminopropionate and N—(C-₁₂-C₁₈-alkyl)-β-iminodipropionate; N-acylamidoalkyl-N,N-dimethylacetobetaine, preferably, N—(C-₈-C₁₈-acyl)-aminopropyl-N,N-dimethyl-acetobetaine; C-₁₂-C₁₈-alkyldimethyl-sulfopropyl-betaine; imidazoline-based amphoteric surfactants (e.g., miranole, or steinafone), preferably, 1-(β-carboxy-methyloxiethyl)-1-(carboxymethyl)-2-lauryl-imidazolium; and amine oxide (e.g., C-₁₂-C₁₈-alkyldimethylamine oxide and fatty acid amido-alkyl-dimethylamine oxide). The amphoteric surfactants may be also present in other formulations, such as hair rinses, hair tonics and hair restorers, and anhydrous oily formulations (e.g., hair oil, hair pomades, and hair brilliants).

The shampoo composition of the present invention also includes a foaming agent such as fatty acid mono- and di-alkaneolamide, which may be exemplified by cocamide MEA (a mixture of coconut acid monoethanolamides having a chemical formula of R—CO—NHCH₂CH₂OH, wherein the R group may be residue remaining after removal of a carboxyl group of a coconut fatty acid), cocamide DEA (a mixture of diethanolamide having a chemical formula of R—CO—N(CH₂CH₂ON), wherein, the R group is the same as above), oleamide MEA, and oleamide DEA.

The shampoo composition of the present invention also includes a thickening agent in order to give viscosity ranging from about 4,000 to about 9,000 cps. Examples of the thickening agent include aryl ester and C₁₀₋₃₀ alkyl acrylate, acryl acid of sucrose and carbopol 1342 as a copolymer of acrylic acid and/or methacrylic acid. The thickening agent useful in the shampoo composition may also include cellulose derivatives, such as hydroxypropyl methylcellolose, hydroxyethyl cellulose, carboxymethyl cellulose, and the like. In addition, a salt, for example, NaCl, may be added in a small amount, and the salt is typically added in an amount of from about 0.25 to 0.6 wt %.

The shampoo composition of the present invention may also include perfumes, coloring agents, opacifiers, conditioners (e.g., polyquaternium-7[polymeric quaternary ammonium salt of acrylamide and dimethyl diaryl ammonium chloride]), and the like, which are standard components in shampoo.

The shampoo composition of the present invention, if necessary, may include acid, base, and buffering agents in order to maintain its pH in the range of from 4 to 10, preferably, 6.5 to 8.0, more preferably 6.9 to 7.4. To minimize cryptogenic skin irritation, even more preferable is neutral pH. Properties of such compounds for controlling pH values and manner of using said compounds are well known in the art.

A better understanding of the present invention may be obtained in light of the following examples which are set forth to illustrate, but are not to be construed to limit the present invention. The following embodiments exemplify toxicity assays of a compound, 4-hydroxy-5-methoxy-4-[2-methyl-3-(3-methyl-2-butenyl)-2-oxiranyl]-1-oxaspiro[2,5]octan-6-one, and pharmaceutical preparations containing the compound as an active ingredient.

### EXAMPLE 1

### Antifungal activity of 4-hydroxy-5-methoxy-4-[2-methyl-3-(3-methyl-2-butenyl)-2-oxiranyl]-1-oxaspiro[2,5]octan-6-one against Pityrosporum ovale

1. Test for Antifungal Activity Against Pityrosporum ovale

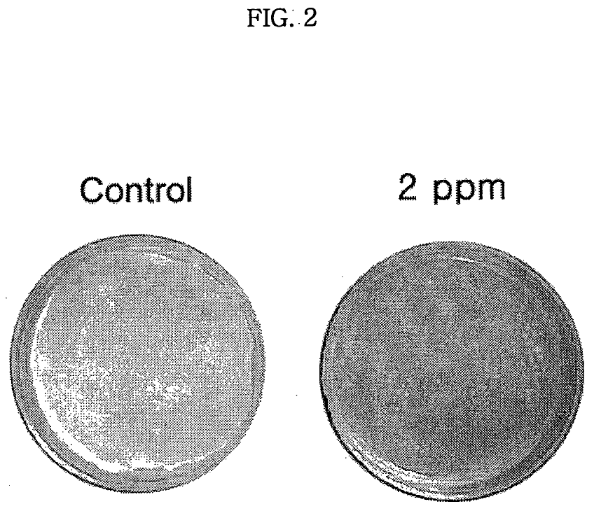

A paper disc diffusion method was used to investigate antifungal activity against a fungus, Pityrosporum ovale. Sabouraud's dextrose solid medium containing 1% of corn oil and 0.1% Tween-80 was aliquotted onto 87 mm plane plates, and allowed to harden. In order to examine antifungal activity against P. ovale, 200% of P. ovale cultured in Sabouraud's dextrose solid medium at 30° C. for 2 days was smeared onto the plates, and then dried. 45 μl of an acetone solution containing 0.1% of 4-hydroxy-5-methoxy-4-[2-methyl-3-(3-methyl-2-butenyl)-2-oxiranyl]-1-oxaspiro[2,5]octan-6-one was aliquated onto paper discs, and, after completely evaporating off the acetone, put onto the P. ovale-smeared medium. Plates not treated with 4-hydroxy-5-methoxy-4-[2-methyl-3-(3-methyl-2-butenyl)-2-oxiranyl]-1-oxaspiro[2,5]octan-6-one were used as controls. Thereafter, incubation was carried out at 30° C. for 3 days, and it was observed whether a growth inhibition area formed around the discs or not. As a result, the compound was found to have antifungal activity against P. ovale (refer to FIG. 2).

### 2. Measurement of Minimum Inhibition Concentration (MIC) of 4-hydroxy-5-methoxy-4-[2-methyl-3-(3-methyl-2-butenyl)-2-oxiranyl]-1-oxaspiro[2,5]octan-6-one Against Pityrosporum ovale

For use in this test, P. ovale was cultured in Sabouraud's dextrose solid medium containing 1% corn oil and 0.1% Tween-80 at 30° C. for 2 days. After Sabouraud's dextrose solid medium containing 1% corn oil and 0.1% Tween-80 was aliquotted onto 6 cm plane plates, 4-hydroxy-5-methoxy-4-[2-methyl-3-(3-methyl-2-butenyl)-2-oxiranyl]-1-oxaspiro[2,5]octan-6-one was added in various concentration. When the medium was hardened, 150 μl of the cultured P. ovale was smeared onto the medium, followed by incubation at 30° C. for 3 to 5 days. Observing with naked eye, a minimum concentration capable of inhibiting the growth of the fungus was determined as MIC (Minimum Inhibition Concentration).

MIC versus P. ovale of 4-hydroxy-5-methoxy-4-[2-methyl-3-(3-methyl-2-butenyl)-2-oxiranyl]-1-oxaspiro[2,5]octan-6-one is given in Table 1, below. As apparent in Table 1, it was found that MIC of 4-hydroxy-5-methoxy-4-[2-methyl-3-(3-methyl-2-butenyl)-2-oxiranyl]-1-oxaspiro[2,5]octan-6-one is below 2 ppm (refer to FIG. 2).

TABLE 1
Active spectrum against P. ovale of 4-hydroxy-5-methoxy-4-[2-methyl-3-
(3-methyl-2-butenyl)-2-oxiranyl]-1-oxaspiro[2,5]octan-6-one

| Fungus | MIC(μg/ml) |
|---|---|
| Pityrosporum ovale | <2 ppm |

### EXAMPLE 2

### Test for Dermal Toxicity of 4-hydroxy-5-methoxy-4-[2-methyl-3-(3-methyl-2-butenyl)-2-oxiranyl]-1-oxaspiro[2,5]octan-6-one

4-hydroxy-5-methoxy-4-[2-methyl-3-(3-methyl-2-butenyl)-2-oxiranyl]-1-oxaspiro[2,5]octan-6-one, which was isolated from the culture filtrate and then purified, was tested for acute dermal toxicity using four-week old female rats, according to the standards provided by the Korean Food & Drug Administration. After the rats were shaved, 4000 mg/kg (body weight) of the compound was uniformly applied to about 10% of body surface area of rats. After observation for 15 days, there was not detected irritation, such as erythema or crusting, indicating that 4-hydroxy-5-methoxy-4-[2-methyl-3-(3-methyl-2-butenyl)-2-oxiranyl]-1-oxaspiro[2,5]octan-6-one is low in toxicity while having antifungal activity.

### EXAMPLE 3

### Shampoo Preparation for Normal Hair

A shampoo was prepared using the ingredients below. An original solution containing 1.64% of carbopol 1342, which was manufactured by uniformly dispersing copolymer powder using a Quadro distributor and then pushing into aqueous vapor under a vacuum condition, and deionized water was put into a vessel, and heated to about 70° C. After surfactants, sodium laureth sulfate and sodium cocoil sarcocinate was added, a foaming agent, cocamide MEA, and a pearlescent agent, ethylene glycol disterate was subsequently added, and completely dissolved. After that, the solution was slightly cooled, an active ingredient, 4-hydroxy-5-methoxy-4-[2-methyl-3-(3-methyl-2-butenyl)-2-oxiranyl]-1-oxaspiro[2,5]octan-6-one was added with stirring. Then, the solution was raised to 40° C., and the solution was supplemented with polyquaternium-7 as a conditioner, quaternium-15 and tetrasodium EDTA as preservatives, a coloring agent, a perfume, and NaCl for regulating viscosity of the solution. The pH of the solution was adjusted between 6.9 and 7.4 by adding a solution of 25% NaOH, and the remainder of the solution was then made up with deionized water, giving a shampoo.

| Ingredient | Wt % |
|---|---|
| Oxaspiro[2,5]oxtane-6-one | 1 |
| Sodium laureth sulfate | 30 |
| Sodium cocoil sarcocinate | 10 |
| Cocamide MEA | 4 |
| Ethylene glycol disterate | 1.25 |
| Polyquaternium-7 | 1 |
| Carbopol 1342 | 0.6 |
| Tetrasodium EDTA | 0.5 |
| Perfume oil | 0.5 |
| Sodium chloride | 0.3 |
| 25% Sodium hyroxide | 0.92 |
| Quaternium-15 | 0.05 |
| Coloring agent | 0.001 |
| Deionized water | Adjusted to 100 |

### EXAMPLE 4

### Shampoo Preparation for Dry or Damaged Hair

Using the below ingredients, a shampoo preparation was prepared according to the same procedure as described in Example 3.

| Ingredient | Wt % |
|---|---|
| Oxaspiro[2,5]oxtane-6-one | 1.5 |
| Sodium laureth sulfate | 30 |
| Sodium cocoil sarcocinate | 10 |
| Cocamide MEA | 4 |
| Ethylene glycol disterate | 1.25 |
| Polyquaternium-7 | 5 |
| Carbopol 1342 | 0.5 |
| Tetrasodium EDTA | 0.5 |
| Perfume oil | 0.5 |
| Sodium chloride | 0.4 |
| 25% Sodium hyroxide | 0.7333 |
| Quaternium-15 | 0.05 |
| Coloring agent | 0.0018 |
| Deionized water | Adjusted to 100 |

In the shampoo preparation of Examples 3 and 4, the addition amount of sodium hyroxide can be slightly modified in order to maintain the pH in the more preferable range from 6.9 to 7.4. Also, the addition amount of sodium chloride can be slightly modified to accomplish a desired viscosity.

### EXAMPLE 5

### Hairdressing Preparation

The below ingredients were put into a vessel to obtain a hairdressing.

| Ingredient | Wt % |
|---|---|
| Oxaspiro[2,5]oxtane-6-one | 1.3 |
| Light mineral oil | 72.0 |
| Isopropyl myristate | 22.0 |
| Lanoline | 2.0 |
| Lanoline ester | 1.5 |
| Perfume | 1.2 |

### EXAMPLE 6

### Hydrophilic Ointment Preparation

A hydrophilic ointment washable with water was prepared using the following ingredients. Stearyl alcohol and white petrolatum were dissolved using steam, and heated to 75° C. Added to water heated to 75° C. in advance were lauryl sulfate, propylene glycol, methylparabene and propylparabene. An active ingredient, 4-hydroxy-5-methoxy-4-[2-methyl-3-(3-methyl-2-butenyl)-2-oxiranyl]-1-oxaspiro[2,5]octan-6-one, was added to a water phase, and mixing was continued to reach coagulation state, generating a ointment.

| Ingredient | Wt % |
|---|---|
| Oxaspiro[2,5]oxtane-6-one | 1.5 |
| White petrolatum | 25 |
| Stearyl alcohol | 25 |
| Propylene glycol | 12 |
| Sodium lauryl sulfate | 1 |
| Methylparabene | 0.025 |
| Deionized water | Adjusted to 100 |

### EXAMPLE 7

### Cream Preparation

A cream of oil-in-water (O/W) type was prepared using the following ingredients. After an oil phase and a water phase were separately heated to 70° C., the oil phase was slowly added to the water phase with stirring to generate a crude emulsion. The emulsion was cooled to about 55° C. and then homogenized, followed by shaking incubation until coagulation, giving a cream.

| Ingredient | Wt % |
|---|---|
| (Oil phase) | |
| Oxaspiro[2,5]oxtane-6-one | 1.5 |
| Stearyl alcohol | 15 |
| Bee wax | 8 |
| Sorbitan monoolate | 1.25 |
| (Water phase) | |
| A sorbitol solution, 70% USP | 7.5 |
| Polysorbate 80 | 3.75 |
| Methylparabene | 0.025 |
| Propylparaben | 0.015 |
| Deionized water | Adjusted to 100 |

### EXAMPLE 8

### Vanishing Cream Preparation

A cream of oil-in-water (O/W) type was prepared using the following ingredients. After, an oil phase and a water phase were separately heated to about 65° C., the oil phase was slowly added to the water phase with stirring to generate a crude emulsion. The emulsion was cooled to about 50° C., and then homogenized, followed by shaking incubation until coagulation, giving a cream.

| Ingredient | Wt % |
|---|---|
| (Oil phase) | |
| Oxaspiro[2,5]oxtane-6-one | 1.5 |
| Stearic acid | 13 |
| Stearyl alcohol | 1 |
| Cetyl alcohol | 1 |
| (Water phase) | |
| Glycerin | 10 |
| Methylparabene | 0.1 |
| Propylparaben | 0.05 |
| Potassium hydroxide | 0.9 |
| Deionized water | Adjusted to 100 |

### EXAMPLE 9

### Gel Preparation

A lubricating jelly was prepared using the following ingredients. The ingredients were dispersed in 40 ml of hot water (80 to 90° C.), and cooled in a refrigerator overnight. Separately, carbopol 934 was dispersed in 20 ml of water, adjusted in pH to 7.0 using a sufficient amount of 1% sodium hydroxide solution, 12 ml of which may be needed per 100 ml, and then supplemented with water to give a final volume of 40 ml. Also, separately, methylparabene and 4-hydroxy-5-methoxy-4-[2-methyl-3-(3-methyl-2-butenyl)-2-oxiranyl]-1-oxaspiro[2,5]octan-6-one were dissolved in propylene glycol. The three solutions were carefully mixed to avoid aeration to generate a gel.

| Ingredient | Wt % |
|---|---|
| Oxaspiro[2,5]oxtane-6-one | 1.5 |
| Metocel 90 H.C. 4000 | 0.8 |
| Carbopol 934 | 0.24 |
| Propylene glycol | 16.7 |
| Sodium hydroxide | Amount required |
| | for pH 7 |
| Deionized water | Adjusted to 100 |

### EXAMPLE 10

### Test for Skin Irritation

A test for skin irritation was performed according to the paper published by Genji Imokawa, et. al., and a closed patch test was conducted using Finn Chamber of Norgesplaster A/S company.

10 μl of the formulations prepared in Examples 3 to 9 were put onto paper discs suitable for Finn Chamber, allowed to absorb, and transferred to Finn Chamber, and then attached onto skin of ten adult males. After 48 hours, the Finn Chamber was removed therefrom, and skin was washed with running water and dried. After 2 hours, skin conditions were evaluated according to the following criteria.

Criteria for the Estimation of Skin Conditions (Irritation Strength)
−: No change with naked eye
±: Slight rubefaction
+: Medium rubefaction
++: Strong rubefaction and edema

The results are shown in Table 2, below.

TABLE 2

| | Skin condition |
|---|---|
| Example 3 | — |
| Example 4 | — |
| Example 5 | — |
| Example 6 | — |
| Example 7 | — |
| Example 8 | — |
| Example 9 | — |

As apparent in Table 2, the dermal preparations of the present invention were found to cause no irritation to skin, and thus have no toxicity.

### EXAMPLE 11

### Test for Inhibitory Effect on Dandruff Formation

Ten male patients aged 20 to 40, having dandruff, were used to test for preventive effects versus dandruff of the present formulations, as follows. After dividing the scalp of each patient into two sides, one side was rinsed with the shampoo prepared in Example 3, which contains 4-hydroxy-5-methoxy-4-[2-methyl-3-(3-methyl-2-butenyl)-2-oxiranyl]-1-oxaspiro[2,5]octan-6-one, and another side with a shampoo without containing 4-hydroxy-5-methoxy-4-[2-methyl-3-(3-methyl-2-butenyl)-2-oxiranyl]-1-oxaspiro[2,5]octan-6-one, which was used as a control. The procedure was repeated once every day for 10 days, and the degree of dandruff was estimated with the naked eye at intervals of 2 days.

Criteria for the estimation are given as five steps, below, and twenty scalp portions in each case were analyzed.
0: No detection of dandruff
1: No detection of dandruff, but slight dandruff by finger rubbing
2: A little dandruff, but less than the control
3: Dandruff in a similar amount to the control
4: Much more dandruff than the control

The above criteria were further simplified according to the grading system in Table 3, below.

TABLE 3

| Estimation | Average |
|---|---|
| O | 0 to 1.5 |
| X | 1.5 to 4.0 |

Note:
“O” means that dandruff is effectively prevented
“X” means that dandruff is not effectively prevented

As a result, the shampoo formulations of the present invention began to be evaluated as “O” starting day 3 of the test, and on the tenth day, skin condition of patients was completely recovered.

### EXAMPLE 12

### Test for Treatment Effect on Damaged Scalp

Patients having severe dandruff have, in most cases, damaged scalp, but, when the conventional dandruff-treating agents are used for treatment, the damaged scalp is very slowly recovered or not. The cream containing 4-hydroxy-5-methoxy-4-[2-methyl-3-(3-methyl-2-butenyl)-2-oxiranyl]-1-oxaspiro[2,5]octan-6-one prepared in Example 9 according to the present invention, and a formulation without the compound, 4-hydroxy-5-methoxy-4-[2-methyl-3-(3-methyl-2-butenyl)-2-oxiranyl]-1-oxaspiro[2,5]octan-6-one, which was used as a control, were applied to damaged scalp, and the degree of recovery was examined. The two formulations were applied by respective halves of scalps of rubbing to ten patients having dandruff as well as damaged scalp, and conditions of the scalp were evaluated every 2 days with the naked eye. Criteria for the estimation are given, below, and twenty scalp portions in each case were analyzed.
0: No detection of damaged scalp
1: Scalp damage reduced by 80% compared to the control
2: Scalp damage reduced by 50% compared to the control
3: Scalp damage reduced by 30% compared to the control
4: No recovery of damaged scalp compared to the control

The above criteria were further simplified according to the grading system Table 4, below.

TABLE 4

| Estimation | Average |
|---|---|
| O | 0 to 1.5 |
| X | 1.5 to 4.0 |

Note:
“O” means that dandruff is effectively prevented
“X” means that dandruff is not effectively prevented

As a result, the cream formulation of the present invention began to be evaluated as “O” from day 5 of the test, and on day 10, damaged scalp of all patients was completely recovered.

Various modifications of the present invention in addition to those shown and described herein will be apparent to those skilled in the art from the foregoing description. Such modifications are also intended to fall within the scope of the appended claims. The foregoing disclosure includes all the information deemed essential to enable those skilled in the art to practice the claimed invention.

### INDUSTRIAL APPLICABILITY

As described above, in accordance with the present invention, the pharmaceutical composition containing the compound, 4-hydroxy-5-methoxy-4-[2-methyl-3-(3-methyl-2-butenyl)-2-oxiranyl]-1-oxaspiro[2,5]octan-6-one, as an active ingredient has an excellent effect of preventing and treating skin conditions caused by the fungus, Pityrosporum ovale. Therefore, the pharmaceutical composition of the present invention may be greatly useful in industrial application.

1. A pharmaceutical composition for the prevention or treatment of seborrhea comprising a pharmaceutically effective amount of 4-hydroxy-5-methoxy-4-[2-methyl-3-(3-methyl-2-butenyl)-2-oxiranyl]-1-oxaspiro[2,5]octan-6-one.

2. The pharmaceutical composition as set forth in claim 1, wherein the seborrhea is dandruff.

3. The pharmaceutical composition as set forth in claim 2, wherein the seborrhea is selected from the group consisting of pityriasis simplex, pityriasis oleosa, pityriasis circinata, seborrheic dermatitis, and acne vulgaris.

4. The pharmaceutical composition as set forth in any one of claims 1 to 3, wherein the composition is in the form of a vanishing cream.

5. The pharmaceutical composition as set forth in any one of claims 1 to 3, wherein the composition is in the form of a shampoo.

6. The pharmaceutical composition as set forth in claim 1, wherein the pharmaceutically effective amount is from 0.01 to 10 wt % of the composition.

7. A method of preventing or treating seborrhea comprising applying a pharmaceutical composition containing a pharmaceutically effective amount of 4-hydroxy-5-methoxy-4-[2-methyl-3-(3-methyl-2-butenyl)-2-oxiranyl]-1-oxaspiro[2,5]octan-6-one to skin of a patient suffering from seborrhea.

8. The method as set forth in claim 7, wherein the seborrhea is dandruff.

9. The method as set forth in claim 7, wherein the seborrhea is selected from the group consisting of pityriasis simplex, pityriasis oleosa, pityriasis circinata, seborrheic dermatitis, and acne vulgaris.

10. The method as set forth in any one of claims 7 to 9, wherein the composition is in the form of a vanishing cream.

11. The method as set forth in any one of claims 7 to 9, wherein the composition is in the form of a shampoo.

12. The method as set forth in claim 7, wherein the pharmaceutically effective amount is from 0.01 to 10 wt % of the composition.
